(19) United States
(12) Patent Application Publication
(10) Pub. No.: US 2019/0167660 A1
(43) Pub. Date: Jun. 6, 2019

(54) PPAR-gamma AGONIST FOR TREATMENT OF BONE DISORDERS

(71) Applicant: InteKrin Therapeutics, Inc., Redwood City, CA (US)

(72) Inventor: Dennis Lanfear, Portola Valley, CA (US)

(21) Appl. No.: 16/326,070

(22) PCT Filed: Aug. 18, 2017
(86) PCT No.: PCT/US2017/047584

§ 371 Date: Feb. 15, 2019

Related U.S. Application Data
(60) Provisional application No. 62/376,732, filed on Aug. 18, 2016.

(51) Int. Cl. A61K 31/47; A61P 19/00

(52) U.S. Cl. CPC A61K 31/47; A61P 19/00

(57) ABSTRACT

Methods of treatment of bone disorders, including osteoporosis or its symptoms, with PPARΥ agonists, and in particular, the compound of formula (I) known as INT131: (I)

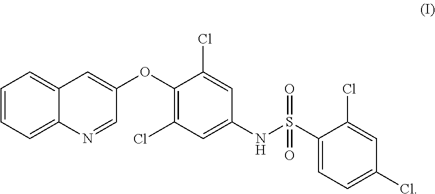 [SMILES: O=S(=O)(Nc1cc(Cl)c(Oc2cnc3ccccc3c2)c(Cl)c1)c1ccc(Cl)cc1Cl]

### FIELD OF THE INVENTION

The present invention relates to methods of treatment of bone disorders, including bone loss and osteoporosis.

### BACKGROUND OF THE INVENTION

Osteoporosis is a disease that results in the weakening of bone and an increase in the risk of fracture. It has been reported that American females over the age of 50 have about a 50% chance of breaking a bone during their lifetime. Osteoporosis is believed to contribute to about 1.5 million fractures a year in the United States, including about 700,000 spinal fractures and about 300,000 hip fractures. According to the Mayo Clinic, about 25% of the people over 50 who fracture a hip die within a year of the incident. The risk of breaking a bone for an osteoporotic individual doubles after the first fracture. The risk of breaking a second vertebra for an osteoporotic individual increases about four-fold after the first spinal fracture.

Osteoporosis is a very common reason for broken bones among the elderly. Because of an aging population, osteoporosis and other bone destructive disorders are a major problem in our health system. Primary osteoporosis treatments focus on decreasing bone destruction by reducing the formation and maturation of osteoclasts. Osteoporosis treatments include estrogen replacement therapy, administration of bisphosphonates, selective estrogen receptor modulators, calcitonin, and antibodies such as denosumab. However, such therapies are sometimes associated with adverse effects, e.g., breast cancer, osteonecrosis of the jaw, hypercalcemia, and hypertension.

Accordingly, there exists a need for new treatments for osteoporosis.

INT131 (also known as CHS-131) is a novel, first-in-class, selective modulator of peroxisome proliferator-activated receptor gamma (PPARγ). The PPARγ is a transcription factor belonging to the steroid/thyroid/retinoid receptor superfamily. To date, PPARγ agonists have been therapeutic agents for disorders such as obesity, diabetes and dyslipidemia.

INT131 is structurally different from other PPARγ agonists. INT131 lacks the TZD (glitazone) scaffold of rosiglitazone and pioglitazone. Therefore, INT131 binds the AF2 (transcriptional activation function 2) helix without contacting helix 12. As a result, INT131 selectively activates PPARγ functions.

PPARγ protein function regulates target gene transcription in a ligand-dependent, cofactor-dependent manner by differential co-factor/co-repressor recruitment. As a result of these complex combinatorial chemistry mechanisms, and the unique structure of INT131, the effects of selective activation of PPARγ is difficult to predict. For instance, it has been shown that subjects who are administered INT131 lack TZD-induced adverse events. Therefore, transcriptional activation effected by INT131 differs from other PPARγ agonists. As a result, the effect of other PPARγ agonists on patients is not predictive of the utility of INT131.

### SUMMARY OF THE INVENTION

It has now been discovered that PPARγ agonist INT131 (also known as CHS-131) is effective for treating osteoporosis.

In one aspect, the present invention provides methods of treating osteoporosis and symptoms thereof. The methods typically involve administering to a subject in need thereof a therapeutically effective amount of compound INT131 described in U.S. Pat. No. 7,601,841. INT131 is unique among PPARγ agonists in that it is a selective activator of a highly limited number of PPARγ pathways. Among these INT131-sensitive pathways are metabolic pathways including those pathways regulated by the hormone adiponectin.

As a result of this selective activation, administration of INT131 to patients results in fewer side effects than administration of other PPARγ agonists. For example, INT131 was equally efficacious in reducing HbA1c levels as 45 mg of pioglitazone but subjects taking INT131 experienced less edema, weight gain, and hemodilution than those taking pioglitazone. See, DePaoli, et al. Diabetes Care. 2014 July; 37(7):1918-23. Thus, INT131 can administered to treat osteoporosis while limiting side effects. Limiting side effects is advantageous as it helps preserve the quality of life for subject taking the medication and results in improved subject compliance with taking medication.

In particular, the invention provides a method of treating osteoporosis or symptoms thereof in a subject in need thereof comprising administering to the subject a pharmaceutical composition comprising a therapeutically effective amount of a compound of formula (I),

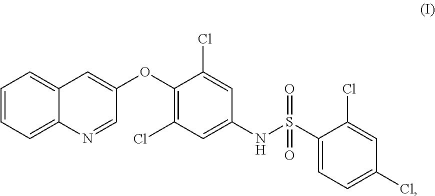 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the compound of formula (I) (i.e, INT131) is provided in the form of a besylate salt.

In one embodiment, the therapeutically effective amount is from about 0.1 to about 10 mg, more preferably from about 1 to about 4 mg, even more preferably from about 2 to about 3 mg, and most preferably about 3 mg.

The pharmaceutical compositions used in the methods of the invention may be administered to the subject twice a day, daily, every other day, three times a week, twice a week, weekly, every other week, twice a month, or monthly.

Preferably, the methods of the invention result in increase of the adiponectin level in the subject by at least about 30%, at least about 68%, at least about 175%, or at least about 200%.

### BRIEF DESCRIPTION OF THE DRAWINGS

FIG. 1 is a bar graph of levels of adiponectin following administration of INT131

### DETAILED DESCRIPTION OF THE INVENTION

In particular, the compound (I),

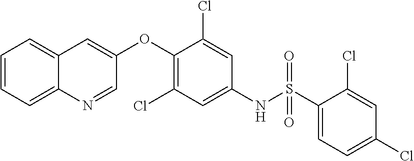 [SMILES: O=S(=O)(Nc1cc(Cl)c(Oc2cnc3ccccc3c2)c(Cl)c1)c1ccc(Cl)cc1Cl]

has been found to be unexpectedly effective for the treatment of osteoporosis. This compound is also known as INT131 and CHS-131.

### Definitions

The terms “treat”, “treating” and “treatment” refer to a method of alleviating or abrogating a disease and/or its attendant symptoms.

The term “therapeutically effective amount” refers to that amount of the compound being administered sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### EMBODIMENTS OF THE INVENTION

A new use of a known compound that modulates PPARγ has now been discovered. Specifically, it has been discovered that PPARγ agonists, and in particular, INT131, are effective to treat osteoporosis.

Thus, in one embodiment, the present invention is directed to a method of treating osteoporosis or its symptoms in a subject in need thereof comprising administering to the subject a pharmaceutical composition comprising a therapeutically effective amount of INT131 or a pharmaceutically acceptable salt, prodrug, or isomer thereof.

In a study in which obese mice were administered 10 mg/kg of INT131, it was suggested that INT131 may be a desirable drug that does not affect bone mass for humans with type 2 diabetes. Lee, et al., Selective PPARγ modulator INT131 normalizes insulin signaling defects and improves bone mass in diet-induced obese mice, Am J Physiol Endocrinol Metab. 2012 Mar 1; 302(5):E552-60. However, it has not been shown that INT131 can be used to treat osteoporosis, treat bone loss, or increase bone growth in human subjects.

Without wishing to be limited to a particular theory, it is believed that INT131 (and other PPARγ agonists) are able to increase adiponectin levels and therefore, treat osteoporosis. The prior art is rather contradictory on the role of adiponectin in osteoporosis. On the one hand, it has been shown that adiponectin stimulates bone formation and remodeling and inhibits bone resorption. Lubkowska et al, Adiponectin as a Biomarker of Osteoporosis in Postmenopausal Women: Controversies, Hindawi Publishing Corporation, Disease Markers, Volume 2014, Article ID 975178, page 2. It has also been shown that adiponectin may induce osteoblasts proliferation and differentiation. Id., page 8. However, at the same time, there have been some studies that showed no abnormalities in bone turnover in mice with adiponectin deficiency or adiponectin overexpression in the liver. Id., citing Shinoda et al, “Regulation of bone formation by adiponectin through autocrine/paracrine and endocrine pathways,” Journal of Cellular Biochemistry, vol, 99, no. 1, pp. 196-208, 2006. The conclusion in the review article is that potential benefits of treating osteoporosis patients by pharmacological regulation of adiponectin are controversial and require further research.

It has also been discovered that INT131 promotes mesenchymal stem cells (MSCs) to differentiate and develop into osteoblasts—the cells that synthesize new bone. At the same time, INT131 does not result in an increase of adipocytes. Since bone loss in osteoporotic patients, and in people with age-dependent bone loss, is associated with increased adipose tissue in the bone marrow, it is advantageous to increase osteoblasts while preventing increase adipocytes (cells that primarily compose adipose tissue).

Administration of INT131 can increase osteoblasts in a subject by at least about 10%, by at least about 20%, by at least about 30%, by at least about 40%, by at least about 50%, by at least about 60%, by at least about 70%, by at least about 80%, by at least about 90%, or by at least about 100%.

Thus, INT131 promotes bone growth and bone healing. This is useful to treat osteoporosis and other aliments where bone growth is desired. Women are most at risk of bone loss and may benefit significantly from INT131.

INT131 induced bone growth or bone healing can treat subjects with various disease and conditions including, but not limited to, osteoporosis, bone fractures, low bone mineral density (BMD), a low-calcium diet, smoking, and hormone changes. Hormone changes may be age-related and may include excess parathyroid hormone, low growth hormone, low estrogen in women (e.g. post-menopausal women and women who stop menstruating, such as athletes and those with anorexia), and low testosterone in men.

Additionally, subject taking certain medications which can cause in bone loss will benefit from INT131 induced bone growth or bone healing. Such medications include, but are not limited to, chemotherapy drugs, aluminum-containing antacids (e.g. Maalox®, Mylanta®, Amphogel®, Gelusil® and Rolaids®), antirejection/immunosuppressive therapy (e.g. cyclosporine and tacrolimus), heparin, loop diuretics (e.g. furosemide and torsemide), medroxyprogesterone acetate, methotrexate, synthetic glucocorticoids (e.g. prednisone, dexamethasone), breast cancer drugs (e.g. aromatase inhibitors anastrozole (Arimidex®), letrozole (Femara®) and exemestane (Aromasin®)), androgen deprivation therapy, proton pump inhibitors (e.g. Prevacid®, Losec®, Pantoloc®, Tecta®, Pariet® and Nexium®), Depo-Provera, thyroid replacement therapy (Synthroid®, Eltroxin®), anti-seizure drugs carbamazepine (e.g. Tegretol® and phenytoin Dilantin®), drugs used to treat high blood pressure (can increase the risk of falls and fractures in older adults), Diuretics (e.g. furosemide (Lasix®)), Alpha adrenergic blockers (e.g. tamsulosin (Flomax®)), acetaminophen (e.g. when taken for a period of at least 3 years), narcotic and opioid medications (e.g. morphine), and medications that cause low vitamin D levels.

The benefits of INT131 (i.e. promoting bone growth and treating or preventing bone loss) is surprising since thiazolidinediones such as pioglitazone and rosiglitazone have been reported to cause bone loss.

Accordingly, it is surprising and unexpected that INT131 can treat osteoporosis. In one embodiment, INT131 treats osteoporosis in men and women. In other embodiment, INT131 treats osteoporosis in postmenopausal women.

Additionally, it is surprising and unexpected that INT131 can treat bone loss. It is additionally surprising and unexpected that INT131 can increase bone growth.

In one embodiment, INT131 is in the form of a besylate salt.

In another embodiment, the therapeutically effective amount is from about 0.1 to about 10 milligrams, preferably from about 0.5 to about 5 milligrams and more preferably from about 1 to about 3 milligrams. In another embodiment, the therapeutically effective amount is at least about 0.5 milligrams, about 1 milligrams, about 2 milligrams, about 3 milligrams, about 4 milligrams, about 5 milligrams, about 6 milligrams, about 7 milligrams, 8 milligrams, about 9 milligrams or about 10 milligrams.

In another embodiment, a composition comprising a therapeutically effective amount of INT131 is administered to a subject in need thereof at an interval that includes, but is not limited to, twice a day, daily, every other day, three times a week, twice a week, weekly, every other week, twice a month, monthly, and every other month.

In one embodiment, administration of INT131 to a subject in need thereof reduces the incidence of bone fractures in the subject as compared to placebo or a standard of care. In another embodiment, the bone fractures are vertebral fractures.

In one embodiment, administration of INT131 to a subject in need thereof increases bone mass of the subject. In another embodiment, administration of INT131 to a subject in need thereof increases the bone mineral density of the subject.

In another embodiment, a composition comprising a therapeutically effective amount of INT131 is administered orally to a subject. In yet another embodiment, the composition is substantially the same as those disclosed in US Publication 2013-0243865, the disclosure of which is expressly incorporated herein by reference.

### EXAMPLES

### Example 1

INT131 is a Potent Upregulator of Adiponectin in Patients with Reduced Adiponectin Levels

### Method

A randomized, double-blind, placebo-controlled, 24-week study was conducted in which adiponectin levels were measured. The study had a 2-week lead-in period, a 24-week double-blind treatment period and a 2-week follow up period. 367 subjects with type 2 diabetes (TD2)—a disease in which patient adiponectin levels are reduced—were randomly assigned to receive either 0.5, 1, 2 or 3 milligrams (“mg”) of INT131 besylate, 45 mg of pioglitazone or placebo daily for 24 weeks. To measure adiponectin levels blood was drawn at Weeks 0, 2, 6, 12 and 24.

The results of this study demonstrated that 1, 2, and 3 mg doses of INT131 caused a statistically significant reduction of HbA_{1c} levels as compared to placebo. Further, the study demonstrated that the 2 and 3 mg doses of INT131 reduced HbA_{1c} levels at least as well as 45 mg of pioglitazone, which is an FDA approved treatment for TD2. See, DePaoli, et al. Diabetes Care 2014; 37:1918-1923. Thus, 2 and 3 mg doses of INT131 would be effective in treating TD2.

### Adiponectin Results

At baseline (Week 0) mean adiponectin levels were 1.94 micrograms per milliliter (“μg/mL”). The mean adiponectin levels at baseline and Week 24, and the mean change in adiponectin levels from baseline (Week 0) to Week 24 are disclosed in Table 1, below. The standard deviation for samples tested in each group is listed in (parenthesis). Mean baseline adiponectin values were similar for the treatment groups.

TABLE 1
Changes in Adiponectin Serum Levels

| Mean | | | | | | 45 mg |
|---|---|---|---|---|---|---|
| Adiponectin | | 0.5 mg | 1 mg | 2 mg | 3 mg | Pi- |
| (μg/mL) | Placebo | INT131 | INT131 | INT131 | INT131 | oglitazone |
| n | 56 | 56 | 59 | 60 | 60 | 57 |
| Week 0 | 1.85 | 1.73 | 1.87 | 1.87 | 2.00 | 2.32 |
| | (1.153) | (1.190) | (1.217) | (1.098) | (1.215) | (2.185) |
| Week 24 | 1.9 | 2.28 | 3.15 | 5.14 | 5.83 | 5.28 |
| | (1.510) | (1.540) | (2.533) | (3.650) | (4.826) | (3.222) |
| Mean | 0.05 | 0.56 | 1.28 | 3.27 | 3.83 | 2.96 |
| Change | (0.680) | (0.906) | (1.882) | (3.002) | (4.313) | (2.618) |

The treatment comparisons of 1 mg, 2 mg, and 3 mg doses of INT131 with placebo were statistically significant (p≤0.0109). This demonstrates that treatment with INT131 resulted in a statistically significant increase in adiponectin levels in patients suffering from a disease in which adiponectin levels are reduced (e.g. TD2). Thus, INT131 is therapeutically effective in treating patients with diseases (e.g. osteoporosis) in which adiponectin levels are reduced.

Additionally, the treatment comparisons of 0.5 mg, 1 mg, and 3 mg doses of INT131 with pioglitazone 45 mg were statistically significant (p≤0.0408). Thus, the dose dependent increase of adiponectin levels by INT131 is independent from the increase resulting from pioglitazone. Conclusions

The effect of treatment on serum adiponectin was assessed, enabling a more direct comparison of the relative potencies of INT131 and pioglitazone 45 mg as selective PPARγ modulators. The mean change in adiponectin from baseline to Week 24 with LOCF (last observation carried forward) was 0.05 μg/mL for the placebo group, 0.56 μg/mL for the INT131 0.5 mg group, 1.28 μg/mL for the INT131 1 mg group, 3.27 μg/mL for the 2 mg group, 3.83 μg/mL for the INT131 3 mg group, and 2.96 μg/mL for the pioglitazone 45 mg group. Therefore, in a manner quantitatively different from the effects on HbA_{1c}, where the INT131 dose roughly equivalent to pioglitazone 45 mg is between 2 mg and 3 mg, a dose of INT131 between 1 mg and 2 mg was equivalent to pioglitazone 45 mg for increasing adiponectin levels.

Surprisingly, administration of INT131 at either 2 or 3 mg resulted in a greater upregulation of serum adiponectin levels than did administration of at least 22 times the amount of pioglitazone. Small amounts of INT131 are at least as efficacious in treating diseases in which adiponectin levels are reduced as are other drugs which also increase adiponectin levels.

Administration of 1, 2, or 3 mg of INT131 treats patients suffering from diseases in which adiponectin levels are reduced.

### Example 2

### INT131 is a Potent Upregulator of Adiponectin in Healthy Subjects

### Method

A study was conducted to determine the effect of INT131 on serum adiponectin levels. Thirty healthy subjects were randomly selected to receive either placebo, 0.1 mg INT131, 1 mg INT131 or 4 mg INT131 daily for 14 days. To measure adiponectin levels blood was drawn at Days 1, 4, 8 and 14.

### Results

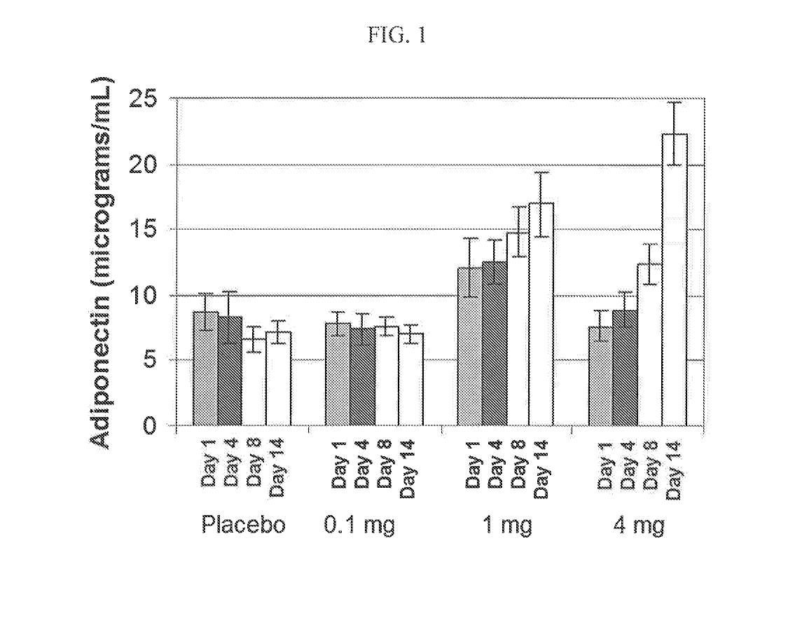

From Day 1 to Day 14 administration of placebo and 0.1 mg INT131 resulted in no significant change in serum adiponectin levels and further administration of 0.1 mg INT131 resulted in no significant change in adiponectin levels over placebo. See FIG. 1. However, administration of 1 mg or 4 mg INT131 resulted in a significant change in serum adiponectin levels over placebo and a significant change from Day 1 to Day 14. Thus, administration of INT131 is capable of upregulating adiponectin in healthy individuals.

### Example 3

### INT131 Activates Bone Remodeling

### Method

A study was conducted to determine the effect of INT131 on serum adiponectin levels. Thirty healthy subjects were randomly selected to receive either placebo, 0.1 mg INT131, 1 mg INT131 or 4 mg INT131 daily for 14 days. To measure adiponectin levels blood was drawn at Days 1, 4, 8 and 14.

### Results

From Day 1 to Day 14 administration of placebo and 0.1 mg INT131 resulted in no significant

What is claimed is:

1. A method of treating osteoporosis in a subject in need thereof comprising administering to the subject a pharmaceutical composition comprising a therapeutically effective amount of a compound of formula (I), 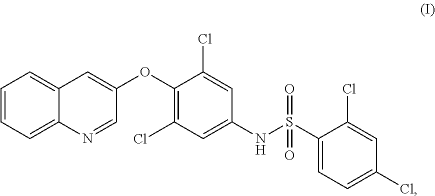 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

2. A method of treating bone loss in a subject in need thereof comprising administering to the subject a pharmaceutical composition comprising a therapeutically effective amount of a compound of formula (I), 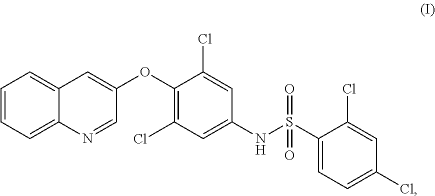 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

3. A method of increasing bone growth in a subject in need thereof comprising administering to the subject a pharmaceutical composition comprising a therapeutically effective amount of a compound of formula (I), 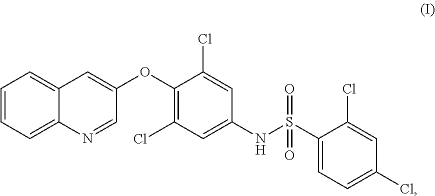 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

4. A method of treating the symptoms of osteoporosis in a subject in need thereof comprising administering to the subject a pharmaceutical composition comprising a therapeutically effective amount of a compound of formula (I), 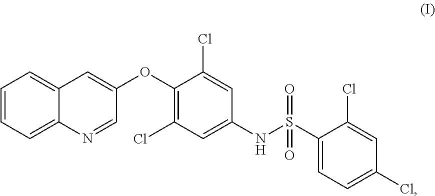 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

5. A method of treating the symptoms of bone loss in a subject in need thereof comprising administering to the subject a pharmaceutical composition comprising a therapeutically effective amount of a compound of formula (I), 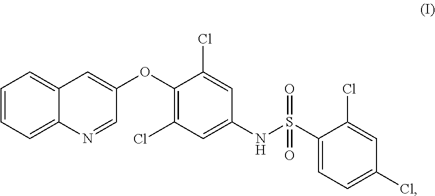 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

6. The method of claim 3, wherein the bone is healed over a shorter time than when the compound is not administered.

7. The method of any one of claims 1 to 5, wherein the compound of formula (I) is in the form of a besylate salt.

8. The method of any one of claims 1 to 5, wherein the therapeutically effective amount is from about 0.1 to about 10 milligrams.

9. The method of claim 8, wherein the therapeutically effective amount is from about 1 to about 4 milligrams.

10. The method of claim 9, wherein the therapeutically effective amount is from about 2 to about 3 milligrams.

11. The method of claim 10, wherein the therapeutically effective amount is about 3 milligrams.

12. The method of any one of claims 1 to 5, wherein the pharmaceutical composition is administered to the subject twice a day, daily, every other day, three times a week, twice a week, weekly, every other week, twice a month, or monthly.

13. The method of claim 12, wherein the pharmaceutical composition is administered to the subject daily.

14. The method of any one of claims 1 to 5, wherein the pharmaceutical composition is administered to the subject daily and the therapeutically effective amount of the compound is about 3 milligrams.

15. The method of any one of claims 1 to 5, wherein the method provides an increase in adiponectin level in the subject by at least about 30%, at least about 68%, at least about 175%, or at least about 200%.

16. The method of claim 15, wherein the increase is by at least about 175%.

17. The method of any one of claims 1 to 5, wherein the method provides an increase of osteoblasts in the subject.

18. The method of any one of claims 1 to 5, wherein the method does not result in an increase in adipocytes or adipose tissue.

19. The method of any one of claims 1 to 5, wherein the method provides an increase of osteoblasts, but not adipocytes or adipose tissue, in the subject.
